(19) United States
(12) Patent Application Publication
(10) Pub. No.: US 2021/0009926 A1
(43) Pub. Date: Jan. 14, 2021

(54) ENZYMATIC PROCESS FOR PRODUCING INTERMEDIATES USEFUL AS ESTERQUAT PRECURSORS

(71) Applicant: EASTMAN CHEMICAL COMPANY, KINGSPORT, TN (US)

(72) Inventors: NEIL WARREN BOAZ, KINGSPORT, TN (US); JENNIFER MICHELLE LLOYD, KINGSPORT, TN (US)

(73) Assignee: EASTMAN CHEMICAL COMPANY, KINGSPORT, TN (US)

(21) Appl. No.: 16/968,805

(22) PCT Filed: Feb. 5, 2019
(86) PCT No.: PCT/US2019/016571

§ 371 Date: Aug. 10, 2020

Related U.S. Application Data
(60) Provisional application No. 62/629,879, filed on Feb. 13, 2018.

(51) Int. Cl. C11D 3/386; C11D 3/30; C11D 1/62; C11D 3/20

(52) U.S. Cl. CPC C11D 3/38627; C11D 3/2093; C11D 1/62; C11D 3/30

(57) ABSTRACT

The invention provides a process for the preparation of a modified triethanolamine/fatty acid ester composition ratio by treatment of a conventional tnethanolamine fatty acid ester composition with a hydrolytic enzyme. Also provided are novel mixtures of tnethanolamine and mono-, di-, and tri-esters of fatty acids which are useful in the preparation of cationic surfactants useful in fabric softening applications. In one aspect, the method increases the triester fraction without significantly affecting the amount of mono-ester and unesterified species, which in turn adds to the flexibility in the formulation of the corresponding fabric softening compositions.

### FIELD OF THE INVENTION

This invention relates to the field of fabric softeners. In particular, it relates to an enzymatic method for preparing intermediates useful in the manufacture of cationic surfactants.

### BACKGROUND OF THE INVENTION

Triethanolamine-based ester quaternary ammonium compounds (“quats”) find widespread use as fabric softening agents in rinse-cycle fabric softeners. These materials are generally prepared from triethanolamine and a fatty acid or fatty acid methyl ester using a deficient amount of fatty acid compared to the available number of hydroxyl groups. This deficient but defined amount of fatty acid is used so that a processable ester quat mixture can be obtained, as these water insoluble materials are generally formulated as water-based emulsions. Thus, the acid-catalyzed esterification results in a statistical distribution of esters (mono-, di-, and tri-ester) along with some unreacted triethanolamine that after quaternization can be readily formulated. However, the deficient amount of fatty acid limits the fatty content of the product, and thus can lead to fabric softening inefficiencies in the balance of competing properties of dispersibility in water versus performance as fabric softening agents. Using higher amounts of fatty acids results in high levels of tri-ester but corresponding low levels of triethanolamine and monoester which makes emulsion formation of the corresponding quat problematic without resorting to additional processing aids.

A method that would modify the ester ratio (and thus the ester quat ratio) to increase the amount of triester while maintaining the triethanolamine and monoester level would be advantageous.

### SUMMARY OF THE INVENTION

The invention is as set forth in the claims. In one embodiment, the invention provides a process for the preparation of a modified triethanolamine/fatty acid ester composition ratio by treatment of a conventional triethanolamine fatty acid ester composition with a hydrolytic enzyme. The conventional triethanolamine ester composition is a mixture of triethanolamine and esters of the Formulae (II), (III), and (I):

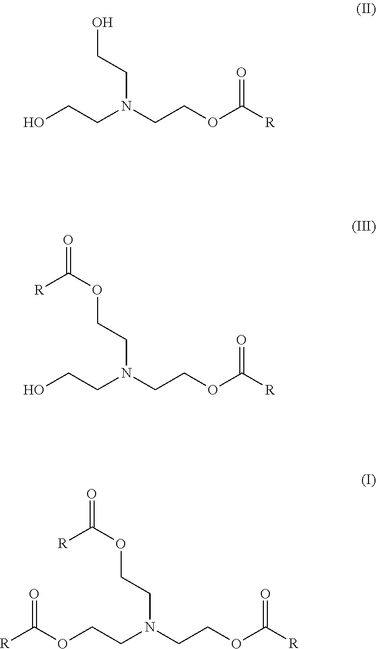

wherein each R is independently chosen from straight and branched-chain, saturated and unsaturated C₁₁-C₂₃ hydrocarbyl groups. The mole ratio of the four components will depend generally upon the stoichiometry of the reaction in which the mixture is formed but is limited by the statistical nature of the product derived from a non-selective acid catalyst. In conventional processes and resulting mixtures, the stoichiometry is generally in the range of 1.5 to 2 equivalents of fatty acid to triethanolamine. The invention as described herein provides a novel enzymatic process to manufacture a novel triethanolamine/fatty acid ester mixture which is more highly desirable for use in making fabric softeners. In particular, increasing the triester content of a conventionally-derived ester quat results in a reduction in mono-ester and unesterified material, which results in an ester quat that is difficult to formulate (see, for example, U.S. Pat. No. 9,388,367), and requires processing aids to afford an adequate dispersion. Thus, the generally accepted opinion that high triester ester quats in general perform poorly. However, a method which increases the triester fraction without significantly affecting the amount of monoester and unesterified species would be of interest, so as to add greater flexibility in the formulation of the corresponding fabric softening compositions.

### DETAILED DESCRIPTION OF THE INVENTION

In one embodiment, the invention provides a method for increasing the molar proportion of triesters of Formula (I)

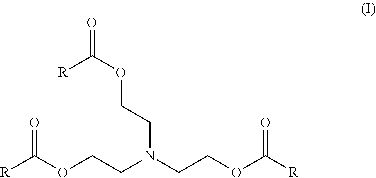

in a mixture comprising (i) triethanolamine and (ii) fatty acid esters having the Formulae (II), (III) and (I):

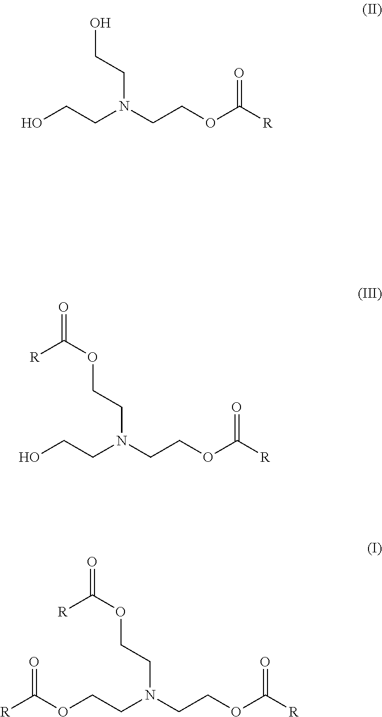

wherein each R is independently chosen from straight and branched-chain, saturated and unsaturated C₁₁-C₂₃ hydrocarbyl groups, which comprises contacting said mixture with a hydrolytic enzyme. In another embodiment, the method results in increasing the molar proportion of triesters of Formula (I) while decreasing the molar proportion of fatty acid esters of Formula (II) and/or (III).

In certain embodiments, treatment of this triethanolamine/fatty acid ester mixture with an enzyme results in a modification of the ester ratio wherein the combined amount of triethanolamine and monoester is relatively unchanged (3 mol % change or less) but the diester is reduced by at least 4 mol % and the triester is increased by at least 4 mol %. Thus, in another embodiment, the invention provides a method for increasing the molar proportion of triesters of Formula (I)

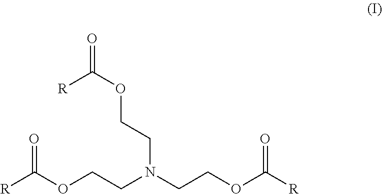

in a mixture comprising (i) triethanolamine and (ii) fatty acid esters having the Formulae (II), (III) and (I):

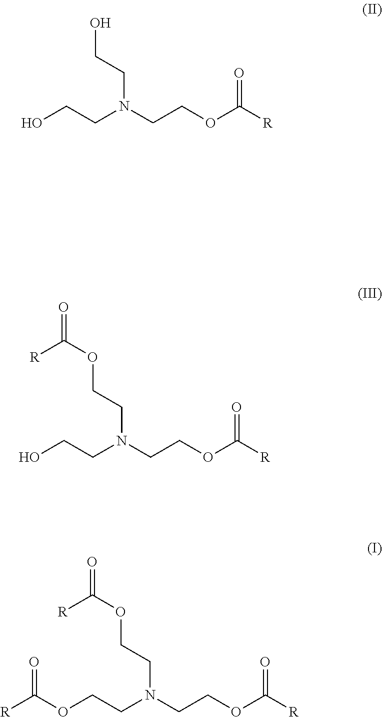

wherein each R is independently chosen from straight and branched-chain, saturated and unsaturated C₁₁-C₂₃ hydrocarbyl groups,
while neither decreasing nor increasing the molar proportion of the combination of triethanolamine and fatty acid esters of Formula (II) more than 3 mole %, while decreasing the fatty acid ester of Formula (III) at least 4 mole % (but no greater than 15 mole %), and increasing the fatty acid ester of Formula (I) by at least 4 mole % (but no more than 15 mole %),
which comprises contacting said mixture with a hydrolytic enzyme.

The mixture thus produced is a novel consequence of the enzymatic equilibration reaction of the triethanolamine/fatty acid ester mixture; this mixture is particularly desirable insofar as the more desirable triester is increased in relative proportion without materially affecting the monoester (and unesterified material). Accordingly, the process provides a novel mixture of triethanolamine/fatty acid esters which are enhanced in triester concentration, and in other embodiments, also decreased in diester as well. Thus, in another embodiment, the invention provides a mixture comprising:
a. triethanolamine in a proportion of about 5 to about 15 mole %, based on the total of a, b, c, and d;
b. a fatty acid monoester of Formula (II)

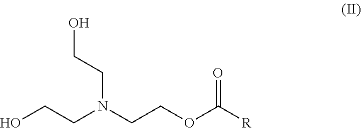

in a proportion of about 22 to about 30 mole %, based on the total of a, b, c, and d;
c. a fatty acid diester of Formula (III)

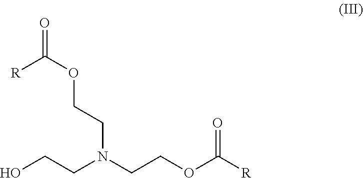

in a proportion of about 38 to about 45 mole % based on the total of a, b, c, and d combined, and
d. a fatty acid triester of Formula (I)

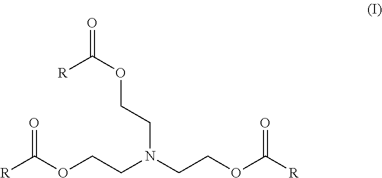

in a proportion of about 17 to about 31 mole %, based on the total of a, b, c, and d combined, wherein the total mole % of a, b, c, and d equals 100%;

wherein each R is independently chosen from straight and branched-chain, saturated and unsaturated C₁₁-C₂₃ hydrocarbyl groups.

In this embodiment, the mixtures may be further characterized as having an average Degree of Substitution (DS) of the hydroxyl groups of the triethanolamine and the esters of the Formulae (II), (III), and (I) from about 1.5 to about 1.9.

In a further embodiment, the invention provides a mixture comprising:
a. triethanolamine in a proportion of about 7 to about 15 mole %, based on the total of a, b, c, and d;
b. a fatty acid monoester of Formula (II)

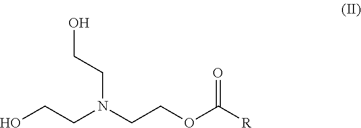

in a proportion of about 23 to about 30 mole %, based on the total of a, b, c, and d;
c. a fatty acid diester of Formula (III)

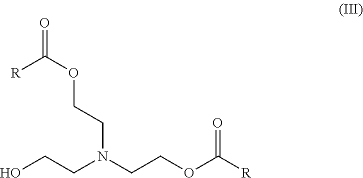

in a proportion of about 38 to about 45 mole % based on the total of a, b, c, and d combined, and
d. a fatty acid triester of Formula (I)

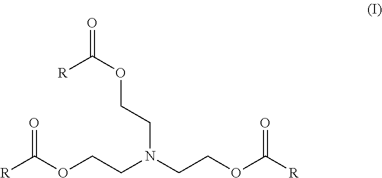

in a proportion of about 17 to about 27 mole %, based on the total of a, b, c, and d combined, wherein the total mole % of a, b, c, and d equals 100%;

wherein each R is independently chosen from straight and branched-chain, saturated and unsaturated C₁₁-C₂₃ hydrocarbyl groups.

In this embodiment, the mixtures may be further characterized as having an average Degree of Substitution (DS) of the hydroxyl groups of the triethanolamine and the esters of the Formulae (II), (III), and (I) from about 1.5 to about 1.75.

The novel mixtures referred to above may then be converted to a variety of subsequent materials as is known in the literature for triethanolamine esters. Of particular interest is alkylation of the nitrogen to afford cationic surfactants. This alkylation can be performed with dialkyl sulfates such as dimethyl sulfate or diethyl sulfate, benzyl halides such as benzyl chloride, and many others as is known in the art. Cationic surfactants derived from triethanolamine esters find their most widespread utility as hair conditioning agents and particularly as rinse-cycle laundry fabric softeners.

Examples of the compounds denoted by Formulae (I), (II), and (III) include those wherein the

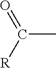

moiety (hereafter “RCO”) is lauroyl, myristoyl, palmitoyl, stearoyl, oleoyl, linoleoyl, or mixtures thereof, or a mixture of C₁₂ to C₂₄ acyl radicals derived from tallow, vegetable, nut, or seed oil. In one embodiment, the compounds of formulae (I), (II), and (III) wherein RCO is a mixture are derived from the fatty acids derived from one or more of palm oil, tallow, and partially hydrogenated tallow.

As used herein, the term “hydrocarbyl” refers to a mono-valent hydrocarbon group. The term includes groups such as alkyls, alkenes, and alkynes. The hydrocarbyl group R may be substituted or unsubstituted; branched or straight-chain; and saturated, mono-unsaturated, or poly-unsaturated. The hydrocarbyl group R may also be a substituted or unsubstituted C₃-C₅ cycloalkyl group.

In one embodiment, R is selected from substituted or unsubstituted, branched- or straight-chain, saturated C₅-C₁₉ alkyl; substituted or unsubstituted, branched- or straight-chain C₅-C₁₇ alkenyl; substituted or unsubstituted, branched- or straight-chain C₅-C₁₇ dienyl; and substituted or unsubstituted C₃-C₅ cycloalkyl.

The hydrocarbyl group of R may be substituted with one to five substituents selected from the group consisting of C₁-C₆ alkoxy, C₁-C₆ carboxyl, C₁-C₁₅ aminocarbonyl, C₁-C₁₅ amido, cyano, C₂-C₆ alkoxycarbonyl, C₂-C₆ alkanoyloxy, hydroxy, aryl, heteroaryl, thioether, C₂-C₁₀ dialkylamino, C₃-C₁₅ trialkylammonium, chlorine, and bromine.

As used herein, the terms “C₁-C₆ alkoxy,” “C₂-C₆ alkoxycarbonyl,” and “C₂-C₆ alkanoyloxy” are used to denote radicals corresponding to the structures —OR², —CO₂R², and —OCOR², respectively, where R² is a substituted or unsubstituted C₁-C₆ alkyl group.

As used herein, the terms “C₁-C₁₅ aminocarbonyl” and “C₁-C₁₅ amido” are used to denote radicals corresponding to the structures —NHCOR³ and —CONHR³, respectively, where R³ is a substituted or unsubstituted C₁-C₁₅ alkyl group.

As used herein, “degree of substitution” or “DS” refers to the overall average of the extent of the hydroxyl moieties of the mixture of triethanolamine and the esters of the Formulae (II), (III), and (I) that exist as ester groups (as the fatty acid esters). For example, the maximum DS would be 3.0 as that would represent a fully esterified triethanolamine substrate, whereas a DS of 2 would indicate an average esterification of two of the three hydroxyl groups on the triethanolamine substrate; similarly, when converted to the corresponding quaternary compounds (“quat”), the DS would correspond to the average esterification on the triethanolamine substrate which would remain intact during the alkylation reaction forming the quat.

The starting mixtures referred to above are prepared using conventional methodology. The mixture is prepared by the esterification of triethanolamine with the chosen (but typically substitutionally deficient, i.e., one typically uses less than three full molar equivalents of fatty acid in the reaction to form the esters) amount of fatty acid to afford a mixture of unreacted triethanolamine, monoester (II), diester (III), and triester (I). This reaction can be performed without catalyst (i.e., self-catalyzed) at high temperatures, or, as is known in the art, with a strong acid catalyst. Generally, high temperatures (up to about 200° C.) with water removal by distillation are used to afford shorter reaction times. A variety of strong acid catalysts have been demonstrated in the art (e.g., U.S. Pat. No. 6,004,913, incorporated herein by reference). Perhaps the most effective catalyst is hypophosphorous acid, as it performs the catalysis at low levels and also minimizes color formation. The strong acid catalyst can remain in the product mixture, or, if desirable, can be removed by base treatment prior to enzymatic equilibration. Minimal residual fatty acid is the result—generally an acid number of less than five.

The ratio of products obtained from this esterification is generally a statistical mixture that depends on the ratio of fatty acid to triethanolamine. The variation of the product composition with respect to starting material ratio is known in the literature, and for convenience the results at various DS levels are presented in Table 1.

TABLE 1
Triethanolamine ester compositions
via acid-catalyzed esterification

| | Normalized molar composition at various | | | |
|---|---|---|---|---|
| | degrees of substitution | | | |
| Component | DS 1.5 | DS 1.6 | DS 1.75 | DS 1.9 |
| Triethanolamine | 10% | 5% | 5% | 2% |
| Monoester 2 | 38% | 35% | 30% | 25% |
| Diester 3 | 42% | 52% | 49% | 50% |
| Triester 1 | 11% | 9% | 16% | 22% |

In one aspect, the method of the invention comprises the enzymatic equilibration of the product mixture from the first step (conventional acid-catalyzed esterification) of the process.

The enzymatic equilibration process of the invention may, in certain embodiments, be carried out at a temperature from about −100° C. and about +100° C., about 20-90° C., or about 50-80° C.

In one embodiment, the enzymatic equilibration process is performed in the absence of significant amounts of solvent. If a solvent is utilized, the solvent can be chosen from cyclic or acyclic ether solvents such as diethyl ether, diisopropyl ether, tert-butyl methyl ether, or tetrahydrofuran, aromatic hydrocarbons such as benzene, toluene, or xylene, aliphatic or alicyclic saturated or unsaturated hydrocarbons such as hexane, heptane, cyclohexane, or limonene, halogenated hydrocarbons such as dichloromethane, dichloroethane, dibromoethane, tetrachloroethylene, or chlorobenzene, polar aprotic solvents such as acetonitrile, dimethyl formamide, or dimethyl sulfoxide, or mixtures thereof.

The enzymatic equilibration process uses a hydrolytic enzyme (for example, a protease, lipase, or esterase enzyme); in one embodiment, the enzyme is a lipase. Examples of these lipases include but are not limited to Lipase PS (from Pseudomonas sp), Lipase PS-C (from Psuedomonas sp immobilized on ceramic), Lipase PS-D (from Pseudomonas sp immobilized on diatomaceous earth), Lipoprime 50T, Lipozyme TL IM, Novozym® 435 (lipase from Candida antarctica immobilized on acrylic resin), Candida antarctica lipase B immobilized on acrylic resin, or Candida antarctica lipase B immobilized on a porous fluoropolymer support as described in U.S. Pat. No. 8,889,373. Immobilized enzymes such as Lipase PS-C, Lipase PS-D, Novozym® 435, Candida antarctica lipase B immobilized on acrylic resin, or Candida antarctica lipase B immobilized on a porous fluoropolymer support allow simple removal of the product from the insoluble supported enzyme and ready re-use of the enzyme.

There are no particular pressure requirements for the enzymatic equilibration process, as no by-products are being removed during this step of the process. The pressure of the reaction is generally around atmospheric pressure, but sub- or super-atmospheric pressures can also be used.

The enzymatic equilibration process affords a significant change in composition compared to the product ratio obtained by the conventional acid-catalyzed esterification. Examples of this change in composition are shown in Table 2.

TABLE 2
Composition comparison between conventional mixtures
and those obtained by the invention for DS 1.6

| | Normalized molar composition | |
|---|---|---|
| Component | (conventional mixture) | (enzymatic equilibration) |
| Triethanolamine | 5% | 10% |
| Monoester 2 | 35% | 27% |
| Diester 3 | 52% | 42% |
| Triester 1 | 9% | 21% |

In certain embodiments, the method of the invention affords at least a 4% decrease in the amount of diester and at least a 5% increase in triester, with the change in some cases being much greater. The upper limit for the change in both the diester and triester is 11 mole % in one embodiment and 15 mole % in another embodiment.

The product of the second step of the process can be converted to a variety of subsequent materials as is known in the literature for triethanolamine esters. Of particular interest is alkylation of the nitrogen to afford cationic surfactants. This alkylation can be performed with dialkyl sulfates such as dimethyl sulfate or diethyl sulfate, benzyl halides such as benzyl chloride, and many others as is known in the art. Cationic surfactants derived from triethanolamine esters find their most widespread utility as hair conditioning agents and particularly as rinse-cycle laundry fabric softeners. See for example, U.S. Pat. Nos. 9,476,012, 5,180,508, 9,338,367, and US 2016/0010029, incorporated herein by reference.

Accordingly, in a further embodiment, the invention provides a mixture comprising:
a. a compound of the formula

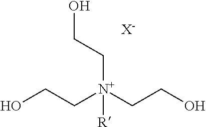

in a proportion of about 5 to 15 about mole %, based on the total of a, b, c, and d;
b. a compound of the formula

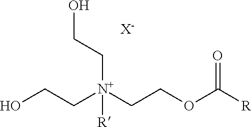

in a proportion of about 22 to about 30 mole %, based on the total of a, b, c, and d;
c. a compound of the formula

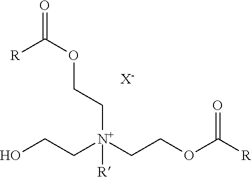

in a proportion of about 38 to about 45 mole % based on the total of a, b, c, and d combined, and
d. a compound of the formula

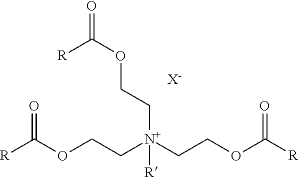

in a proportion of about 17 to about 23 mole %, based on the total of a, b, c, and d combined, wherein the total mole % of a, b, c, and d equals 100%;
wherein each R is independently chosen from straight and branched-chain, saturated and unsaturated C₁₁-C₂₃ hydrocarbyl groups; wherein R′ is a C₁-C₆ alkyl group or a benzyl group and X⁻ is chosen from C₁-C₆ monoalkylsulfate and halide anions.

In this embodiment, the mixtures may be further characterized as having an average DS of the mixture from about 1.5 to about 1.9. In another embodiment, the DS of said mixture is from about 1.5 to about 1.75; a. is present in a proportion of about 7 to about 15%, b. is present in a proportion of about 23 to about 30%, c. is present in a proportion of about 38 to about 45%, and d. is present in a proportion of about 17 to about 27%, based on the total moles of a., b., c., and d., combined, the total equaling 100%.

### EXPERIMENTAL SECTION

### Example 1: Enzymatic Equilibration of the Ester of Triethanolamine with Tallow Fatty Acid (1.6 Equiv) without Acid Catalyst Removal

Step 1: Conventional Acid-Catalyzed Esterification

Triethanolamine (15 g; 0.101 mol) was added to tallow fatty acid (Twin Rivers Technology; TRT-11)(43.9 g; 0.176 mol; 1.6 equiv). A 50% water solution of hypophosphorous acid (0.22 mL; 0.27 g; 0.002 equiv) was added and the mixture was heated with stirring to 140° C. with a headspace nitrogen purge to remove the generated water. After 12 h the reaction was complete and was cooled to 70° C.

Step 2: Equilibration Using Novozym® 435

Novozym® 435 (1.40 g) was added to the above mixture at 70° C. The mixture was stirred at 70° C. under a nitrogen blanket for 12 hours, and the enzyme was removed by filtration. The composition of the ester after Step 1 and Step 2 is shown below.

TABLE 3

Normalized molar composition

| | Step 1 | Step 2 (enzymatic |
|---|---|---|
| Component | (conventional) | equilibration) |
| Triethanolamine | 5% | 10% |
| Monoester 2 | 35% | 27% |
| Diester 3 | 52% | 42% |
| Triester 1 | 9% | 21% |

### Example 2: Enzymatic Equilibration of the Ester of Triethanolamine with Tallow Fatty Acid (1.5 Equiv) without Acid Catalyst Removal

This example used an identical procedure to Example 1 with triethanolamine (15 g; 0.101 mol), tallow fatty acid (Twin Rivers Technology; TRT-11)(41.2 g; 0.151 mol; 1.5 equiv), 50% hypophosphorous acid (0.22 mL; 0.27 g; 0.002 equiv), and Novozym® 435 (1.35 g). The results are shown in Table 4 below.

TABLE 4

Normalized molar composition

| | Step 1 | Step 2 (enzymatic |
|---|---|---|
| Component | (conventional) | equilibration) |
| Triethanolamine | 10% | 15% |
| Monoester 2 | 38% | 30% |
| Diester 3 | 42% | 38% |
| Triester 1 | 11% | 17% |

### Example 3: Enzymatic Equilibration of the Ester of Triethanolamine with Tallow Fatty Acid (1.75 Equivalents) without Acid Catalyst Removal

This example used an identical procedure to Example 1 with triethanolamine (15 g; 0.101 mol), tallow fatty acid (Twin Rivers Technology; TRT-11)(48.0 g; 0.176 mol; 1.75 equiv), 50% hypophosphorous acid (0.22 mL; 0.27 g; 0.002 equiv), and Novozym® 435 (1.50 g). The results are shown in Table 5 below.

TABLE 5

Normalized molar composition

| | Step 1 | Step 2 (enzymatic |
|---|---|---|
| Component | (conventional) | equilibration) |
| Triethanolamine | 5% | 9% |
| Monoester 2 | 30% | 26% |
| Diester 3 | 49% | 39% |
| Triester 1 | 16% | 27% |

### Example 4: Enzymatic Equilibration of the Ester of Triethanolamine with Tallow Fatty Acid (1.9 Equiv) without Acid Catalyst Removal

This example used an identical procedure to Example 1 with triethanolamine (15 g; 0.101 mol), tallow fatty acid (Twin Rivers Technology; TRT-11)(52.2 g; 0.191 mol; 1.9 equivalents), 50% hypophosphorous acid (0.22 mL; 0.27 g; 0.002 equivalents), and Novozym® 435 (1.59 g). The results are shown in Table 6 below.

TABLE 6

Normalized molar composition

| | Step 1 | Step 2 (enzymatic |
|---|---|---|
| Component | (conventional) | equilibration) |
| Triethanolamine | 2% | 5% |
| Monoester 2 | 25% | 22% |
| Diester 3 | 50% | 41% |
| Triester 1 | 22% | 31% |

### Example 5: Enzymatic Equilibration of the Ester of Triethanolamine with Tallow Fatty Acid (1.75 Equiv) without Acid Catalyst Removal

Step 1: Conventional Acid-Catalyzed Esterification

Triethanolamine (15 g; 0.101 mol) was added to tallow fatty acid (Twin Rivers Technology; TRT-11)(48.0 g; 0.176 mol; 1.75 equivalents). A 50% water solution of hypophosphorous acid (0.22 mL; 0.27 g; 0.002 equivalents) was added and the mixture was heated with stirring to 140° C. with a headspace nitrogen purge to remove the generated water. After 12 h the reaction was complete and was cooled to 70° C.

Step 2: Equilibration Using Lipase from Candida antarctica B Immobilized on Acrylic Resin

An immoblized enzyme catalyst (1.50 g) prepared by treatment of the lipase from Candida antarctica B (Novozymes Lipozyme® CaIB-L) with an acrylic resin (Lanxess Lewitat® VP OC 1600) was added to the above mixture at 70° C. The mixture was stirred at 70° C. under a nitrogen blanket for 6 hours, and the enzyme was removed by filtration. The composition of the ester after Step 1 and Step 2 is shown below.

TABLE 7

Normalized molar composition

| | Step 1 | Step 2 (enzymatic |
|---|---|---|
| Component | (conventional) | equilibration) |
| Triethanolamine | 5% | 8% |
| Monoester 2 | 32% | 27% |
| Diester 3 | 50% | 42% |
| Triester 1 | 14% | 23% |

### Example 6: Enzymatic Equilibration of the Ester of Triethanolamine with Tallow Fatty Acid (1.75 Equivalents) without Acid Catalyst Removal

Step 1: Conventional Acid-Catalyzed Esterification

Triethanolamine (15 g; 0.101 mol) was added to tallow fatty acid (Twin Rivers Technology; TRT-11)(48.0 g; 0.176 mol; 1.75 equiv). A 50% water solution of hypophosphorous acid (0.22 mL; 0.27 g; 0.002 equiv) was added and the mixture was heated with stirring to 140° C. with a headspace nitrogen purge to remove the generated water. After 12 h the reaction was complete and was cooled to 70° C.

Step 2: Equilibration Using Lipase from Candida antarctica B Immobilized on Fluoropolymer

Candida antarctica lipase B (Novozymes Lipozyme® CaIB-L) immobilized on a fluoropolymer sheet (270 cm²) cut into small pieces was added to the above mixture at 70° C. The mixture was stirred at 70° C. under a nitrogen blanket for 6 hours, and the enzyme was removed by filtration. The composition of the ester after Step 1 and Step 2 is shown below.

TABLE 8

Normalized molar composition

| | Step 1 | Step 2 (enzymatic |
|---|---|---|
| Component | (conventional) | equilibration) |
| Triethanolamine | 4% | 7% |
| Monoester 2 | 30% | 26% |
| Diester 3 | 50% | 45% |
| Triester 1 | 16% | 22% |

### Example 7: Enzymatic Equilibration of the Ester of Triethanolamine with Tallow Fatty Acid (1.75 Equivalents) Prepared without Acid Catalyst

Step 1: Conventional Non-Catalyzed Esterification

Triethanolamine (324.7 g; 2.18 mol) was added to tallow fatty acid (Twin Rivers Technology; TRT-11)(1039.7 g; 3.81 mol; 1.75 equiv). The mixture was heated with stirring to 140° C. with a headspace nitrogen purge to remove the generated water until the reaction was complete (indicated by an acid number <5).

Step 2: Equilibration Using Lipase from Candida antarctica B Immobilized on Acrylic Resin

A portion of the above triethanolamine fatty acid esters (59.9 g) was treated with 1.50 g of an immobilized Candida antarctica B lipase (Novozymes Lipozyme® CaIB-L) on an acrylic resin (Purolite Lifetech™ ECR1030M) at 70° C. The mixture was stirred at 70° C. under a nitrogen blanket for 3 hours. The composition of the ester after Step 1 and Step 2 is shown below.

TABLE 9

Normalized molar composition

| | Step 1 | Step 2 (enzymatic |
|---|---|---|
| Component | (conventional) | equilibration) |
| Triethanolamine | 6% | 9% |
| Monoester 1 | 31% | 28% |
| Diester 2 | 44% | 39% |
| Triester 3 | 19% | 25% |

### Example 8: Enzymatic Equilibration of the Ester of Triethanolamine with Tallow Fatty Acid (1.75 Equivalents) with Acid Catalyst Removal

Step 1: Conventional Acid-Catalyzed Esterification with Acid Removal Using a Basic Resin

Triethanolamine (324.7 g; 2.18 mol) was added to tallow fatty acid (Twin Rivers Technology; TRT-11)(1039.7 g; 3.81 mol; 1.75 equiv). The mixture was heated with stirring to 140° C. with a headspace nitrogen purge to remove the generated water. After 12 h the reaction was complete and was cooled to 70° C. and treated with dried Amberlyst A-21 weakly basic ion exchange resin (2.40 g). This mixture was stirred for 15 min and the Amberlyst resin was removed by filtration.

Step 2: Equilibration Using Lipase from Candida antarctica B Immobilized on Acrylic Resin

The triethanolamine fatty acid ester mixture prepared above was treated with 1.50 g of an immobilized Candida antarctica B lipase (Novozymes Lipozyme® CaIB-L) on an acrylic resin (Purolite Lifetech™ ECR1030M) at 70° C. The mixture was stirred at 70° C. under a nitrogen blanket for 6 hours. The composition of the ester after Step 1 and Step 2 is shown below.

TABLE 10

Normalized molar composition

| | Step 1 | Step 2 (enzymatic |
|---|---|---|
| Component | (conventional) | equilibration) |
| Triethanolamine | 4% | 8% |
| Monoester 1 | 33% | 28% |
| Diester 2 | 50% | 42% |
| Triester 3 | 14% | 22% |

### Example 9: Enzymatic Equilibration of the Ester of Triethanolamine with Oleic Acid (1.75 Equivalents) without Acid Catalyst Removal

Step 1: Conventional Acid-Catalyzed Esterification

Triethanolamine (20 g; 0.134 mol) was added to oleic acid (66.3 g; 0.235 mol; 1.75 equiv). A 50% water solution of hypophosphorous acid (0.29 mL; 0.35 g; 0.002 equiv) was added and the mixture was heated with stirring to 140° C. with a headspace nitrogen purge to remove the generated water. After 12 h the reaction was complete and was cooled to 70° C.

Step 2: Equilibration Using Novozym® 435

Novozym® 435 (2.05 g) was added to the above mixture at 70° C. The mixture was stirred at 70° C. under a nitrogen blanket for 8 hours, and the enzyme was removed by filtration. The composition of the ester after Step 1 and Step 2 is shown below.

TABLE 11

Normalized molar composition

| | Step 1 | Step 2 (enzymatic |
|---|---|---|
| Component | (conventional) | equilibration) |
| Triethanolamine | 4% | 9% |
| Monoester 2 | 30% | 23% |
| Diester 3 | 50% | 40% |
| Triester 1 | 16% | 27% |

### Example 10: Enzymatic Equilibration of the Ester of Triethanolamine with Palmitic Acid (1.75 Equivalents) without Acid Catalyst Removal

Step 1: Conventional Acid-Catalyzed Esterification

Triethanolamine (15 g; 0.101 mol) was added to palmitic acid (45.1 g; 0.176 mol; 1.75 equiv). A 50% water solution of hypophosphorous acid (0.22 mL; 0.27 g; 0.002 equiv) was added and the mixture was heated with stirring to 140° C. with a headspace nitrogen purge to remove the generated water. After 12 h the reaction was complete and was cooled to 70° C.

Step 2: Equilibration Using Novozym® 435

Novozym® 435 (1.42 g) was added to the above mixture at 70° C. The mixture was stirred at 70° C. under a nitrogen blanket for 6 hours, and the enzyme was removed by filtration. The composition of the ester after Step 1 and Step 2 is shown below.

TABLE 12

Normalized molar composition

| | Step 1 | Step 2 (enzymatic |
|---|---|---|
| Component | (conventional) | equilibration) |
| Triethanolamine | 3% | 7% |
| Monoester 2 | 29% | 24% |
| Diester 3 | 51% | 42% |
| Triester 1 | 16% | 27% |

TABLE 13
This table illustrates the relative compositions of two
embodiments of the novel mixtures of the invention.

| | | | | | | After enzyme | | | |
|---|---|---|---|---|---|---|---|---|---|
| | | Conventional | | | | treatment | | | |
| Example | DS | TEA | Mono | Di | Tri | TEA | Mono | Di | Tri |
| 1 | 1.6 | 5 | 35 | 52 | 9 | 10 | 27 | 42 | 21 |
| 2 | 1.5 | 10 | 38 | 42 | 11 | 15 | 30 | 38 | 17 |
| 3 | 1.75 | 5 | 30 | 49 | 16 | 9 | 26 | 39 | 27 |
| 5 | 1.75 | 5 | 32 | 50 | 14 | 8 | 27 | 42 | 23 |
| 6 | 1.75 | 4 | 30 | 50 | 16 | 7 | 26 | 45 | 22 |
| 7 | 1.75 | 6 | 31 | 44 | 19 | 9 | 28 | 39 | 25 |
| 8 | 1.75 | 4 | 33 | 50 | 14 | 8 | 28 | 42 | 22 |
| 9 | 1.75 | 4 | 30 | 50 | 16 | 9 | 23 | 40 | 27 |
| 10 | 1.75 | 3 | 29 | 51 | 16 | 7 | 24 | 42 | 27 |
| 4 | 1.9 | 2 | 25 | 50 | 22 | 5 | 22 | 41 | 31 |

### Example 11: Synthesis of N-Methyl (Triethanolammonium Tallow Fatty Acid Ester) Methosulfate (TEA Ester Quat) from Enzyme-Equilibrated Ester of Triethanolamine with Tallow Fatty Acid (1.5 Equivalents)

To a 300 ml three neck flask equipped with thermocouple, overhead stirrer and addition funnel was added the ester prepared as in Example 2 (120 g, 0.2148 mol) followed by 16.28 g of isopropanol. The resulting mixture was stirred at a controlled rate and heated to 50° C. using a heating mantle. Dimethyl sulfate (26.55 g, 0.2105 mol) was added dropwise into the TEA ester mixture at a rate such that the internal temperature did not exceed 60° C. After completion of the addition, the mixture was maintained at 60° C. for 7.5 hours at which time all of the dimethyl sulfate was consumed as indicated by ¹H NMR analysis. Free amine titration showed 98% of the TEA ester had been quaternized. The final product contained 90% of N-methyl (triethanolammonium tallow fatty acid ester) methosulfate (TEA ester quat) and 10% IPA.

The invention has been described in detail with particular reference to preferred embodiments thereof, but it will be understood that variations and modifications can be effected within the spirit and scope of the invention.

1. A method for increasing the molar proportion of tri-esters of Formula (I) 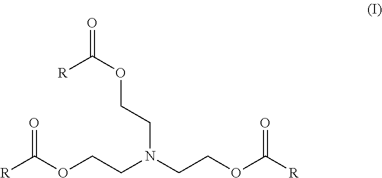
in a mixture comprising (i) triethanolamine and (ii) fatty acid esters having the Formulae (II), (III) and (I): 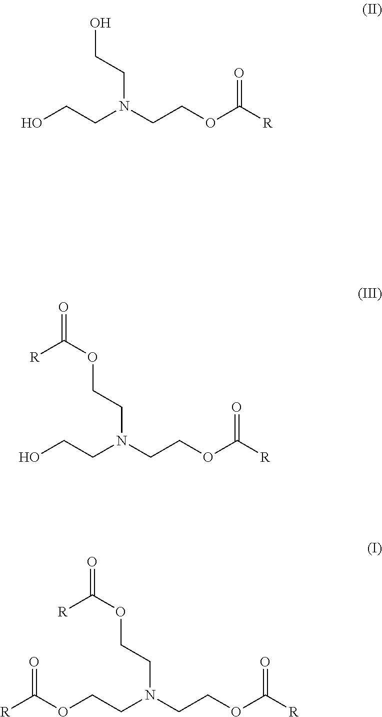
wherein each R is independently chosen from straight and branched-chain, saturated and unsaturated C₁₁-C₂₃ hydrocarbyl groups, which comprises conducting an acid-catalyzed esterification process to obtain the mixture of unreacted triethanolamine, monoester of formula (II), diester of formula (III) and trimester of formula (I), and then contacting said mixture with a hydrolytic enzyme.

2. The method of claim 1, wherein the hydrolytic enzyme comprises one or more of a protease enzyme, a lipase enzyme, or an esterase enzyme.

3. The method of claim 1, wherein the hydrolytic enzyme is chosen from Lipase PS (from Pseudomonas sp), Lipase PS-C (from Psuedomonas sp immobilized on ceramic), Lipase PS-D (from Pseudomonas sp immobilized on diatomaceous earth), Lipoprime 50T, Lipozyme TL IM, Novozym® 435 (lipase from Candida antarctica immobilized on acrylic resin), Candida Antarctica lipase B immobilized on acrylic resin, and Candida antarctica lipase B immobilized on a porous fluoropolymer.

4. The method of claim 1, wherein each R is independently selected from straight and branched-chain, saturated and unsaturated C₁₃-C₁₇ hydrocarbyl groups.

5. The method of claim 1, wherein R in the —C(O)—R moiety is chosen from lauroyl, myristoyl, palmitoyl, stearoyl, oleoyl, linoleoyl, or mixtures thereof.

6. The method of claim 1, wherein RC(O)— is a mixture of C₁₂ to C₂₄ acyl radicals derived from oils chosen from tallow, nut, and seed oil.

7-11. (canceled)

12. A mixture comprising:
a. triethanolamine in a proportion of about 5 to about 15 mole %, based on the total of a, b, c, and d;
b. a fatty acid monoester of Formula (II) 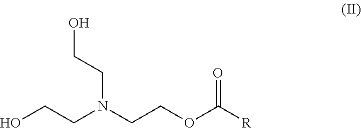
in a proportion of about 22 to about 30 mole %, based on the total of a, b, c, and d;
c. a fatty acid diester of Formula (III) 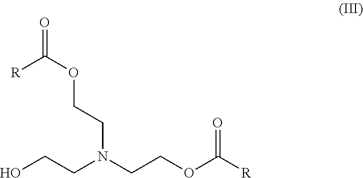
in a proportion of about 38 to about 45 mole % based on the total of a, b, c, and d combined, and
d. a fatty acid triester of Formula (I) 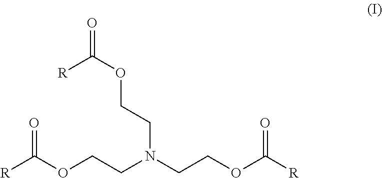
in a proportion of about 17 to about 31 mole %, based on the total of a, b, c, and d combined, wherein the total mole % of a, b, c, and d equals 100%;
wherein each R is independently chosen from straight and branched-chain, saturated and unsaturated C₁₁-C₂₃ hydrocarbyl groups.

13. The mixture of claim 12, wherein the Degree of Substitution (DS) of the hydroxyl groups of the triethanolamine and the esters of the Formulae (II), (III), and (I) is from about 1.5 to about 1.9.

14. The mixture according to claim 12, comprising:
a. triethanolamine in a proportion of about 7 to about 15 mole %, based on the total of a, b, c, and d;
b. a fatty acid monoester of Formula (II) 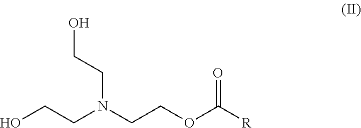
in a proportion of about 23 to about 30 mole %, based on the total of a, b, c, and d;
c. a fatty acid diester of Formula (III) 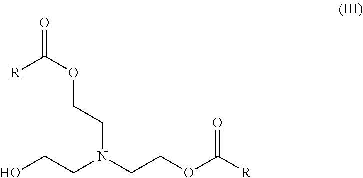
in a proportion of about 38 to about 45 mole % based on the total of a, b, c, and d combined, and
d. a fatty acid triester of Formula (I) 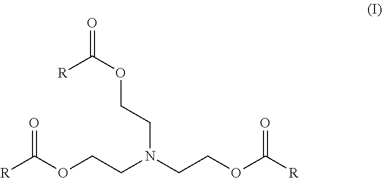
in a proportion of about 17 to about 27 mole %, based on the total of a, b, c, and d combined, wherein the total mole % of a, b, c, and d equals 100%;
wherein each R is independently chosen from straight and branched-chain, saturated and unsaturated C₁₁-C₂₃ hydrocarbyl groups.

15. The mixture of claim 14, wherein the Degree of Substitution (DS) of the hydroxyl groups of the triethanolamine and the esters of the Formulae (II), (III), and (I) is from 1.5 to 1.75.

16. The mixture of claim 14, wherein the RC(O) moiety is a derived from a C₁₆-C₁₈ fatty acid mixture attained from hard and soft Tallow Fatty Acid.

17. A mixture comprising:
a. a compound of the formula 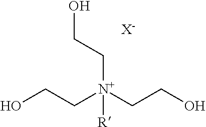
in a proportion of about 5 to 15 about mole %, based on the total of a, b, c, and d;
b. a compound of the formula 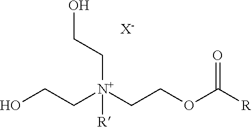
in a proportion of about 22 to about 30 mole %, based on the total of a, b, c, and d;
c. a compound of the formula 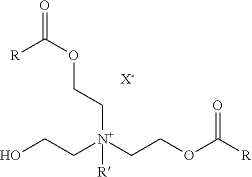
in a proportion of about 38 to about 45 mole % based on the total of a, b, c, and d combined, and
d. a compound of the formula 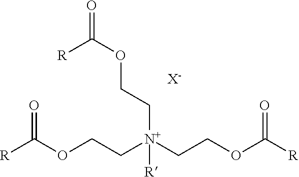
in a proportion of about 17 to about 31 mole %, based on the total of a, b, c, and d combined, wherein the total mole % of a, b, c, and d equals 100%; wherein each R is independently chosen from straight and branched-chain, saturated and unsaturated C₁₁-C₂₃ hydrocarbyl groups; wherein R′ is a C₁-C₆ alkyl group or a benzyl group and X⁻ is chosen from C₁-C₆ monoalkylsulfate and halide anions.

18. The mixture according to claim 17 comprising:
a. a compound of the formula 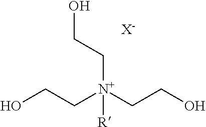
in a proportion of about 7 to about 15 mole %, based on the total of a, b, c, and d;
b. a compound of the formula 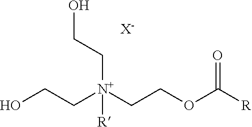
in a proportion of about 23 to about 30 mole %, based on the total of a, b, c, and d;
c. a compound of the formula 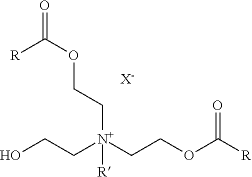
in a proportion of about 38 to about 45 mole % based on the total of a, b, c, and d combined, and
d. a compound of the formula 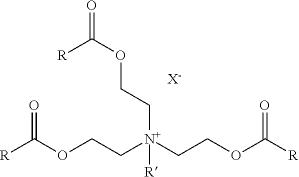
in a proportion of about 17 to about 27 mole %, based on the total of a, b, c, and d combined, wherein the total mole % of a, b, c, and d equals 100%; wherein each R is independently chosen from straight and branched-chain, saturated and unsaturated C₁₁-C₂₃ hydrocarbyl groups; wherein R′ is a C₁-C₆ alkyl group or a benzyl group and X⁻ is chosen from C₁-C₆ monoalkylsulfate and halide anions.

19. The mixture of claim 17, wherein the RC(O) moiety is derived from a C₁₆-C₁₈ fatty acid mixture obtained from hard and soft Tallow Fatty Acid.
